(19) United States
(12) Patent Application Publication
(10) Pub. No.: US 2026/0062318 A1
(43) Pub. Date: Mar. 5, 2026

(54) WATER MONITORING SYSTEM WITH CALCIUM HARDNESS MEASUREMENT

(71) Applicant: ZODIAC POOL SYSTEMS LLC, Carlsbad, CA (US)

(72) Inventor: Anthony Levacque, Carlsbad, CA (US)

(73) Assignee: ZODIAC POOL SYSTEMS LLC, Carlsbad, CA (US)

(21) Appl. No.: 19/284,641

(22) Filed: Jul. 29, 2025

Related U.S. Application Data
(60) Provisional application No. 63/688,735, filed on Aug. 29, 2024.

(51) Int. Cl. C02F 1/00; C02F 1/461; C02F 1/467; C02F 103/42; G01N 33/18

(52) U.S. Cl. CPC C02F 1/008; C02F 1/46104; C02F 1/4674; G01N 33/1853; C02F 2103/42; C02F 2201/4613; C02F 2209/055; C02F 2303/14

(57) ABSTRACT

A water quality monitoring system for a swimming pool or spa includes a saltwater chlorinator and a water sensing device. The water sensing device may measure water hardness of the water of the swimming pool or spa. The saltwater chlorinator may be automatically controlled based on the water hardness measurement from the water sensing device.

### REFERENCE TO RELATED APPLICATION

This application claims the benefit of and priority to U.S. Provisional Patent Application No. 63/688,735, filed on Aug. 29, 2024, and entitled WATER MONITORING SYSTEM WITH CALCIUM HARDNESS MEASUREMENT, the content of which is hereby incorporated by reference in its entirety.

### FIELD OF THE INVENTION

The invention relates to water testing or monitoring systems, and more particularly, but not necessarily exclusively, to water testing or monitoring systems for swimming pools or spas.

### BACKGROUND

Maintaining water quality is important for swimming pools, spas, hot tubs, and other water containing vessels (hereinafter “swimming pools or spas”) to avoid issues for users of the pool as well as equipment of the pool. For example, if the water chemistry of the swimming pool or spa is off, a health hazard may be posed to users and/or operation of various pool equipment and/or systems may be compromised. Conventional testing of water, whether using chemistry kits, remote testing, or maintenance service calls, measures water parameters of pH, chlorine, total chlorine, total alkalinity, and/or cyanuric acid.

Such conventional water parameters are generally useful for controlling water quality, but other untested water parameters may also affect water quality and/or performance of pool equipment. One such water parameter is calcium hardness of the pool water, which may change over time due to multiple factors such as the addition of new chemicals (e.g., calcium hypochlorite increasing a calcium concentration, etc.) and/or a change in the water composition (e.g., due to evaporation, refilling the pool with fresh water from a different source, etc.). Calcium hardness may have a major impact on some pool equipment. As an example, if the calcium hardness is high, a saltwater chlorinator cell will calcify quickly, thereby requiring more frequent cleaning of the saltwater chlorinator cell, but the frequent cleaning of the saltwater chlorinator cell adversely impacts the useful life of the saltwater chlorinator cell. As a further example, if the calcium hardness is high, a quartz tube of a UV system may require more frequent cleaning due to a deposit of scale on an external surface of the quartz tube, which is proportional to the calcium hardness.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain embodiments, a water quality monitoring system includes a water sensing device configured to automatically measure one or more water parameter, and the one or more water parameters includes at least water hardness.

According to some embodiments, a water quality monitoring system may receive and/or obtain a water hardness measurement, and automatically generate an output response based on the water hardness measurement.

According to various embodiments, a water quality monitoring system includes a water sensing device and a saltwater chlorinator. The water sensing device may automatically measure at least water hardness, and the saltwater chlorinator may be automatically controlled based on a water hardness measurement from the water sensing device.

According to some embodiments, a method includes receiving or obtaining a water hardness measurement and generating an output response based on the water hardness measurement.

According to certain embodiments, a non-transitory computer readable storage medium includes a plurality of instructions executable by one or more processors, the plurality of instructions including instructions which, when executed by the one or more processors, cause the one or more processors to perform actions including receiving or obtaining a water hardness measurement and generating an output response based on the water hardness measurement.

Various implementations described in the present disclosure can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 illustrates a pool system according to embodiments.

FIG. 2 illustrates communication between components of the pool system of FIG. 1 according to embodiments.

FIG. 3 illustrates a method according to embodiments.

FIG. 4 illustrates a method according to embodiments.

FIG. 5 illustrates a method according to embodiments.

FIG. 6 illustrates a method according to embodiments.

FIG. 7 illustrates a method according to embodiments.

### DESCRIPTION OF THE INVENTION

Described herein are water monitoring systems and methods with water hardness as a measured water parameter. As used herein, water hardness may refer to calcium hardness or total hardness. Calcium hardness generally refers to the concentration of calcium ions in the water, and total hardness generally refers to the sum of calcium and magnesium ions in the water. Additionally, or alternatively, described herein are systems and methods for adjusting one or more service and/or operating parameters on one or more pieces of pool equipment based on a water hardness measurement. In some embodiments, the systems and methods described herein may utilize a water hardness measurement as a basis for adjusting a polarity reversal on a saltwater chlorinator cell. In various embodiments, the systems and methods described herein may automatically adjust and/or control one or more service parameters of a piece of pool equipment, such as but not limited to a polarity reversal frequency of a saltwater chlorinator cell, based on the water hardness measurement.

In some embodiments, the systems and methods described herein may automatically adjust and optimize a polarity reversal frequency of a saltwater chlorinator cell based on a life of the saltwater chlorinator cell and a cleanliness of the saltwater chlorinator cell.

In various embodiments, the systems and methods described herein may generate an alert and/or otherwise inform a user of a cleanliness of a quartz tube of a UV system and/or adjust a frequency at which cleaning of the quartz tube is needed.

Compared to traditional approaches in which users do not measure water hardness (and thus do not account for the impact of calcium hardness on various pool equipment), the systems and methods described herein automatically measure water hardness. Additionally, or alternatively, compared to traditional approaches, the systems and methods described herein may automatically adjust one or more service and/or operating parameters of a piece of pool equipment, thereby improving performance and the useful life of the pool equipment. Various other benefits and advantages may be realized with the systems, devices, and methods provided herein, and the aforementioned advantages should not be considered limiting.

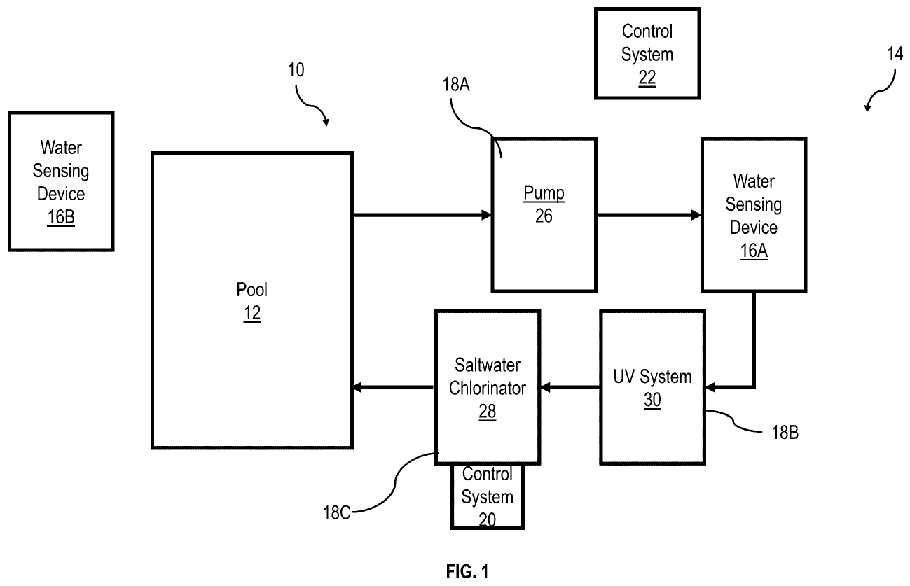

FIG. 1 illustrates an example of a pool system 10 according to embodiments. The pool system 10 generally includes a pool or spa 12 (hereinafter “pool 12”) and a monitoring system 14 for measuring one or more water parameters of water of the pool 12. In addition to the monitoring system 14, the pool system 10 may include one or more pieces of pool equipment 18. Non-limiting examples of pool equipment 18 may include a pump, a filter, a heater, a saltwater chlorinator, a sanitation system (such as but not limited to a UV system), combinations thereof, and/or as otherwise desired. Optionally, a piece of pool equipment 18 may include a controller or control system 20 onboard and/or otherwise associated with the particular piece of pool equipment 18 for controlling operation of the particular piece of pool equipment 18. However, in other embodiments, pool equipment 18 need not include an onboard and/or associated control system 20, and the pool equipment 18 optionally may be controlled by a control system 22 of the pool system 10 and/or the monitoring system 14.

FIG. 1 illustrates three pieces of pool equipment 18A-C where pool equipment 18A is a pump 26, pool equipment 18B is a UV system 28, and pool equipment 18B is a saltwater chlorinator 30. The particular arrangement and location of the pool equipment 18A-C in FIG. 1 should not be considered limiting. Moreover, while three pieces of equipment 18A-C are illustrated in FIG. 1, in other embodiments, any number of pool equipment 18 and/or types or combinations of types of pool equipment 18 may be utilized as desired.

As mentioned, the monitoring system 14 may be utilized to measure one or more water parameters. In certain embodiments, the water parameters measured by the monitoring system 14 may include, but are not limited to, water hardness (e.g., calcium hardness and/or total hardness), pH, free chlorine, total chlorine, total alkalinity, cyanuric acid concentration. In some embodiments, the monitoring system 14 may be utilized to measure at least water hardness.

In some embodiments, the monitoring system 14 may include one or more water sensing devices 16 for measuring one or more water parameters. The water sensing devices 16 may automatically measure one or more water parameters and/or may facilitate manual (or other) measurement of one or more water parameters by a user. In FIG. 1, water sensing device 16A is an automatic water sensing device and water sensing device 16B is a manual water sensing device 16. However, the number, type, and location of water sensing devices 16 of the monitoring system 14 should not be considered limiting. Various types of automatic and/or manual water sensing devices 16 may be utilized as desired. As non-limiting examples, the water sensing devices 16 may be reagent-based water sensing devices, probe-based water sensing devices, combinations thereof, and/or as otherwise desired. In one non-limiting example, the water sensing device 16 may utilize liquid reagents for measuring one or more water parameters such as but not limited to water hardness.

In some embodiments, one or more water sensing devices 16 (e.g., water sensing device 16B) may be mobile and/or movable within the pool system 10. Additionally, or alternatively, one or more water sensing devices 16 (e.g., water sensing device 16A) may be at a fixed and/or predetermined location within the pool system 10 and/or such that the water measurement is performed at a predetermined location within the pool system 10. In one non-limiting example, an automatic water sensing device 16 may be provided inline with one or more systems or equipment of the pool system 10 as illustrated in FIG. 1. In such embodiments, water from the pool may flow through the water sensing device 16. As a further non-limiting example, a reagent-based automatic water sensing device 16 may be provided inline with one or more systems or equipment of the pool system 10, such as but not limited to inline with a water circulation system of the pool system 10.

As mentioned, in some embodiments, the pool system 10 and/or the monitoring system 14 includes the control system 22. The control system 22 may include one or more processing units and/or one or more memory devices. The processing unit may be various suitable processing devices or combinations of devices including but not limited to one or more application specific integrated circuits, digital signal processors, digital signal processing devices, programmable logic devices, field programmable gate arrays, processors, controllers, micro-controllers, microprocessors, other electronic units, and/or a combination thereof. The one or more memory devices may be any machine-readable medium that can be accessed by the processor, including but not limited to any type of long term, short term, volatile, nonvolatile, or other storage medium, and is not to be limited to any particular type of memory or number of memories, or type of media upon which memory is stored. Moreover, as disclosed herein, the term “storage medium,” “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to, portable or fixed storage devices, optical storage devices, wireless channels, and/or various other storage mediums capable of storing that contain or carry instruction(s) and/or data.

In certain embodiments, the control system 22 optionally includes an associated user interface, including but not limited to a graphical user interface or a human machine interface, such that the control system 22 may obtain information from a user and/or provide information to the user. In such embodiments, the user interface and/or human machine interface may be on the control system 22 itself or may be at a location remote from the control system 22.

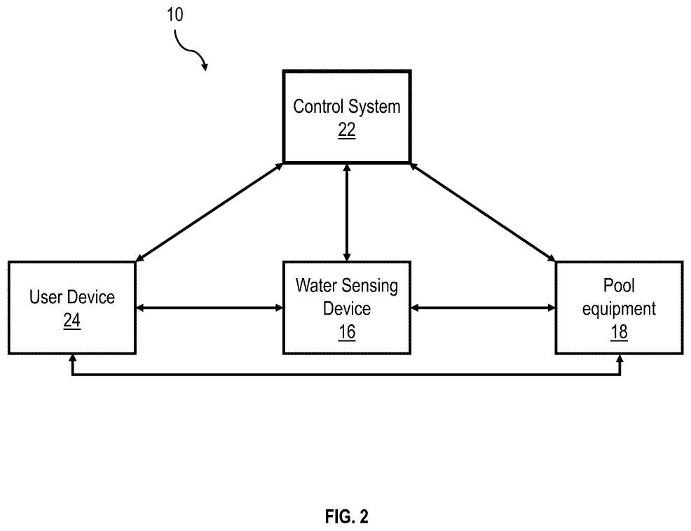

In various embodiments, and with reference to FIG. 2, the various components of the pool system 10 (and/or sub-combinations thereof) may be communicatively coupled (represented by arrows in FIG. 2). In these embodiments, information, such as but not limited to water hardness measurement information, may be directly or indirectly communicated between various components of the pool system 10. Such communication, whether direct or indirect, may utilize various communication techniques as desired, such as but not limited to wired communication and/or wireless communication (e.g., Bluetooth, Li-Fi, LoRa, radio frequency, cellular, NFC, Wi-Fi, etc.).

As a non-limiting example, the system 22 may be communicatively coupled to one of the water sensing devices 16 and one or more pieces of pool equipment 18. In this example, the control system 22 may obtain or receive at least a water hardness measurement from the water sensing device 16 and provide an output response to the pool equipment 18 (e.g., provide the water hardness measurement and/or a control signal based on the water hardness measurement to the pool equipment 18). Additionally, or alternatively, the control system 22 may be communicatively coupled to a user device 24, such as but not limited to a phone, tablet, computer, and/or other device as desired, optionally running an application, and the control system 22 may obtain or receive at least a water hardness measurement from the water sensing device 16 and generate an output response for the user device 24. Additionally, or alternatively, the water sensing device(s) 16, piece(s) of pool equipment 18, and/or user device(s) 24 may directly communicate with each other. As a non-limiting example, one of the water sensing devices 16 may directly communicate at least the water hardness measurement to the pool equipment 18 and/or may directly communicate at least the water hardness measurement to the user device 24. Various other communication and between various groups of components may be utilized using the pool system 10 described herein, and the aforementioned examples should not be considered limiting.

As mentioned, in certain embodiments, the monitoring system 14 described herein advantageously may automatically measure water hardness of water of the pool 12. In these embodiments, the one or more water sensing devices 16 may measure or may be controlled to measure (e.g., by the control system 22) a water hardness of the pool water at various frequencies or intervals as desired. As non-limiting examples, the water hardness may be automatically measured based on a schedule, on-demand, and/or responsive to a trigger event (e.g., a reset of a piece of equipment, an operational status of a piece of equipment, etc.). Compared to traditional approaches, the automatic measurement of water hardness may ensure that the water hardness is measured, optionally allowing for various controls or other output responses as discussed in detail below.

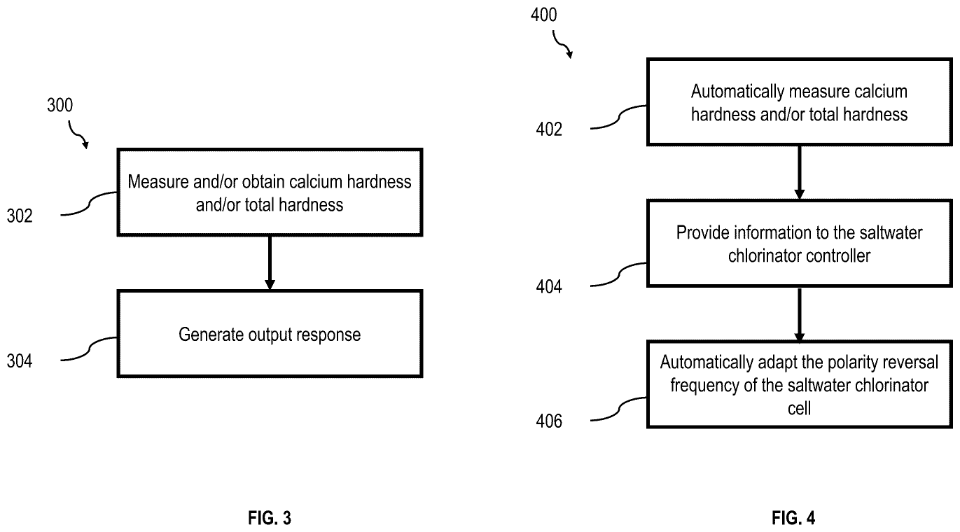

Additionally, or alternatively, and with reference to FIG. 3, the monitoring system 14 advantageously may automatically generate various output responses based on a measured water hardness. The method 300 illustrated in FIG. 3 may be implemented by the control system 22 and/or any other control device or system as desired.

In a block 302, the method 300 includes measuring and/or obtaining a water hardness measurement. In some embodiments, block 302 includes automatically measuring the water hardness using an automatic water sensing device 16. Additionally, or alternatively, block 302 may include receiving a water hardness measurement which was manually (or otherwise) obtained. In such examples, the control system 22 may obtain the manually-measured water hardness measurement via an input device of the control system 22, the user device 24, and/or as otherwise desired.

In a block 304, the method 300 includes generating one or more output responses based on the measured water hardness. Output responses in block 304 may include, but are not limited to, controlling and/or adjusting an operating parameter of one or more pieces of equipment 18, controlling and/or adjusting a maintenance parameter of one or more pieces of equipment 18, and/or generating an alert or notification to a user.

As one non-limiting example, the pool equipment 18 may be the saltwater chlorinator 30, and block 304 includes automatically controlling and/or adjusting an operating parameter of polarity reversal frequency of a saltwater chlorinator cell based on the measured water hardness. As an example, the saltwater chlorinator cell may periodically switch its polarity to prevent and/or reduce any build up of calcium on cell plates of the saltwater chlorinator cell, and block 304 includes adjusting the frequency that the change in polarity is made. As non-limiting examples, a relatively low water hardness measurement may be used to decrease the frequency of the polarity reversal (i.e., adjusted such that there is more time between such changes) while a relatively high water hardness measurement may be used to increase the frequency of the polarity reversal. Optionally, the control of the polarity reversal frequency may be based on additional information, such as but not limited to a life or current operational status of the saltwater chlorinator cell, and block 304 may include controlling the polarity reversal frequency based on operational status and the water hardness. Various other controls of a saltwater chlorinator 30 may be implemented as desired.

As another non-limiting example, the pool equipment 18 may be the UV system 28, and block 304 includes automatically controlling and/or adjusting a maintenance parameter of a frequency of quartz tube cleaning based on the measured water hardness. As an example, UV lamps of the UV system 28 may be housed in a quartz tube, and if the quartz tube becomes dirty (e.g., due to scale deposition), the ability to transmit UV rays from the UV lamp is diminished. In these embodiments, scale deposition on the quartz tube is proportional to the water hardness, and block 304 may include increasing the frequency the quartz tube cleaning based on a relatively high water hardness measurement, and decreasing the frequency of the quartz tube cleaning based on a relatively low water hardness measurement. Various other controls of the UV system 28 may be implemented as desired.

As another non-limiting example, regardless of the type of pool equipment 18, block 304 may include generating an alert or notification to a user. As non-limiting examples, block 304 may include generating an alert on a user interface of the control system 22, generating an alert on the user device 24, combinations thereof, and/or as otherwise desired. Alerts may be visual alerts, auditory alerts, physical alerts (e.g., vibrations), combinations thereof, and/or as otherwise desired. In some embodiments, the alerts may provide the current water hardness measurement, the water hardness measurement relative to a threshold, recommended action items for the user based on the water hardness measurement, recommended action items for pool equipment 18 associated with water hardness, combinations thereof, and/or other information or combinations of information as desired.

FIGS. 4-7 illustrate non-limiting examples of specific implementations 400, 500, 600, 700 of the method 300 of FIG. 3. While these examples are discussed separately below, these processes may be combined and/or otherwise used in any combination or sub-combination thereof as desired. Moreover, various other processes may be utilized alone or in combination using the monitoring system 14 as desired.

Referring to FIG. 4, in a block 402, a method 400 may include automatically measuring water hardness using one or more water sensing devices 16.

In a block 404, the method 400 includes providing the water hardness measurement directly to the control system 20 of the saltwater chlorinator 30. Block 404 may include communicating the water hardness measurement from the water sensing device 16 to the saltwater chlorinator 30 using various wired and/or wireless communication as desired.

In a block 406, the method 400 may include controlling and/or adjusting, by the control system 20 of the saltwater chlorinator 30, the polarity reversal frequency of the saltwater chlorinator cell of the saltwater chlorinator 30.

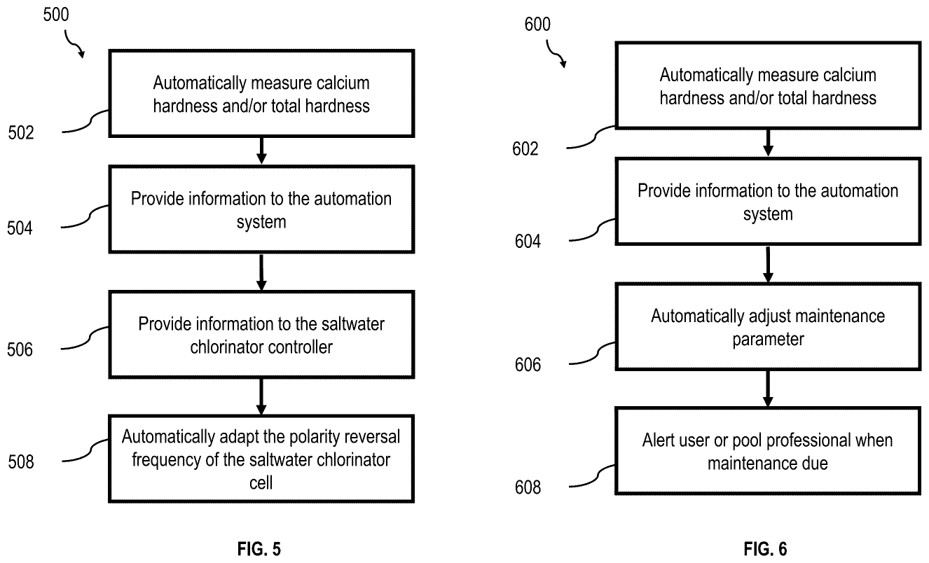

Referring to FIG. 5, in a block 502, a method 500 may include automatically measuring water hardness using one or more water sensing devices 16.

In a block 504, the method 500 includes providing the water hardness measurement to the control system 22.

In a block 506, the method 500 includes providing, by the control system 22, information to the control system 20 of the saltwater chlorinator 30. The communication in blocks 504 and 506 may be various wired and/or wireless communication as desired. In certain embodiments, the information conveyed in block 506 may include the water hardness measurement, control information determined by the control system 22 based on the water hardness measurement. As a non-limiting example, in block 506, the control system 22 may determine an adjusted polarity reversal frequency based on the water hardness, and the information conveyed in block 506 may be the adjusted polarity reversal frequency.

In a block 508, the method 500 may include controlling and/or adjusting, by the control system 20 of the saltwater chlorinator 30, the polarity reversal frequency of the saltwater chlorinator cell of the saltwater chlorinator 30. In various embodiments, depending on the information provided in block 506, block 508 optionally may include determining, by the control system 20 of the saltwater chlorinator 30, an adjusted polarity reversal frequency.

Thus, compared to the method 400 with direct communication between the water sensing device 16 and the saltwater chlorinator 30 as the pool equipment 18, the method 500 includes indirect communication of water hardness information between the water sensing device 16 and the saltwater chlorinator 30.

Referring to FIG. 6, in a block 602, a method 600 may include automatically measuring water hardness using one or more water sensing devices 16.

In a block 604, the method 600 includes providing the water hardness measurement to the control system 22.

In a block 606, the method 600 includes automatically adjusting, by the control system 22, a maintenance parameter of one or more pieces of pool equipment 18. As a non-limiting example, the pool equipment 18 may be the UV system 28, and block 606 includes automatically adjusting a frequency at which a quartz tube of the UV system 28 is cleaned.

In a block 608, the method 600 includes generating an alert or notification to a user when maintenance of the pool equipment 18 is due based on the modified parameter from block 606. In some embodiments, block 608 includes providing the alert on the user device 24, on a user interface of the control system 22, and/or as otherwise desired. In some embodiments, generating the alert includes providing an adjusted maintenance schedule to the user. Additionally, or alternatively, block 608 includes sending a maintenance alert to the user at the frequency determined in block 606 (e.g., the alert is sent monthly if block 606 modifies the frequency of quartz tube cleaning to monthly). The communication in blocks 604, 606, and 608 may be various wired and/or wireless communication as desired.

As illustrated with method 600, some implementations of method 300 may include automatically measuring water hardness and generating a plurality of output responses based on the water hardness measurements.

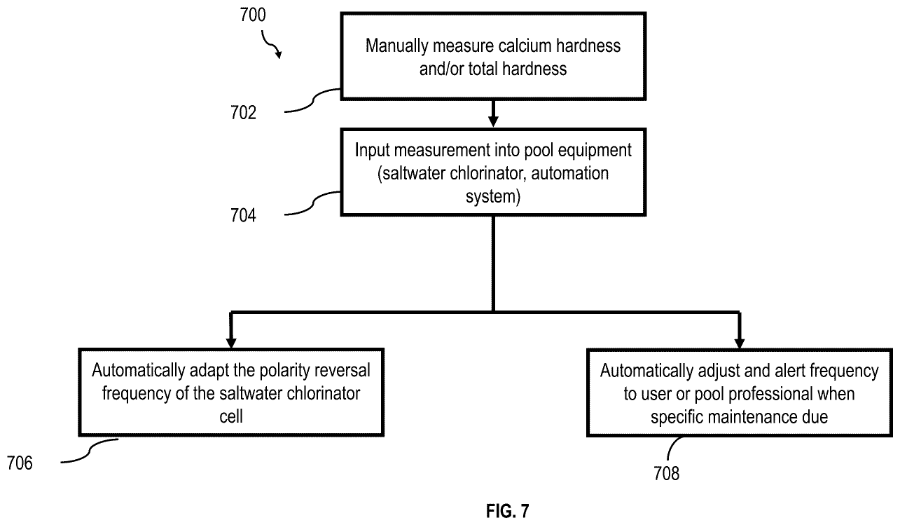

Referring to FIG. 7, in a block 702, a method 700 may include manually measuring water hardness, optionally using one or more water sensing devices 16. Additionally, or alternatively, block 702 may include automatically obtaining water hardness measurements in some embodiments.

In a block 704, the method 700 includes providing the water hardness obtained in block 702 to pool equipment 18 and/or the control system 22. Block 704 may include providing the water hardness using the user device 24 (optionally using an application), using a user interface of the control system 22, and/or using a user interface of the pool equipment 18. As a non-limiting example, the pool equipment 18 may be the saltwater chlorinator 30, and block 704 may include providing the water hardness obtained in block 702 to the saltwater chlorinator 30.

In a block 706, the method 700 optionally includes controlling and/or adjusting, by the control system 20 of the saltwater chlorinator 30, the polarity reversal frequency of the saltwater chlorinator cell of the saltwater chlorinator 30. In some embodiments, an adjusted polarity reversal frequency may be determined by the control system 20 of the saltwater chlorinator 30 (e.g., as discussed regarding method 400) or may be determined by the control system 22 (e.g., as discussed regarding method 500).

Additionally, or alternatively, in a block 708, the method optionally includes automatically adjusting, by the control system 22, a maintenance parameter of one or more pieces of pool equipment 18 and/or generating an alert or notification to a user when maintenance of the pool equipment 18 is due based on a modified maintenance parameter. As a non-limiting example, the pool equipment 18 may be the UV system 28, and block 708 includes automatically adjusting a frequency at which a quartz tube of the UV system 28 is cleaned and/or generating an alert or notification to the user when maintenance of the pool equipment 18 is due based on the modified parameter.

As illustrated with method 700, some implementations of method 300 may include manually measuring water hardness and generating a plurality of output responses for a plurality of pieces of pool equipment 18 based on the water hardness measurements.

As mentioned, methods 400, 500, 600, 700 are non-limiting examples of specific implementations of method 300 and are provided for illustrative purposes. Other process and/or combinations of processes may be realized using the systems and methods described herein.

Various other benefits and advantages may be realized with the systems, devices, and methods provided herein, and the aforementioned advantages should not be considered limiting.

Exemplary concepts or combinations of features of the invention may include:
A. A water quality monitoring system comprising a water sensing device inline with a pool and configured to automatically measure one or more water parameters, wherein the one or more water parameters comprises at least water hardness.
B. The water quality monitoring system of any preceding or subsequent statement or combination of statements, wherein the water sensing device is configured to measure the one or more water parameters using liquid reagent.
C. The water quality monitoring system of any preceding or subsequent statement or combination of statements, wherein water from the pool flows through the water sensing device.
D. A pool comprising local water hardness monitoring.
E. A water quality monitoring system configured to:
   i. receive and/or obtain a water hardness measurement; and
   ii. automatically generate an output response based on the water hardness measurement.
F. The water quality monitoring system of any preceding or subsequent statement or combination of statements, wherein the output response comprises an alert or notification on a user device.
G. The water quality monitoring system of any preceding or subsequent statement or combination of statements, wherein the output response comprises control of a maintenance parameter of piece of pool equipment.
H. The water quality monitoring system of any of any preceding or subsequent statement or combination of statements, wherein the output response comprises control of an operating parameter of piece of pool equipment.
I. The water quality monitoring system of any preceding or subsequent statement or combination of statements, wherein the pool equipment comprises a saltwater chlorinator cell.
J. A water quality monitoring system comprising:
   i. a water sensing device configured to automatically measure at least water hardness; and
   ii. a saltwater chlorinator, wherein the saltwater chlorinator is automatically controlled based on a water hardness measurement from the water sensing device.
K. The water quality monitoring system of any preceding or subsequent statement or combination of statements, wherein the saltwater chlorinator is automatically controlled by controlling a polarity reversal frequency of a saltwater chlorinator cell of the saltwater chlorinator.
L. The water quality monitoring system of any preceding or subsequent statement or combination of statements, wherein the water sensing device and the saltwater chlorinator are configured to directly communicate with each other.
M. The water quality monitoring system of any preceding or subsequent statement or combination of statements, wherein the one or more water parameters comprises water hardness.
N. The water quality monitoring system of any preceding or subsequent statement or combination of statements, further comprising a control system communicatively connected to the water sensing device and the saltwater chlorinator, wherein the control system is configured to:
   i. receive the water hardness measurement from the water sensing device;
   ii. determine a control response for the saltwater chlorinator based on the received water hardness measurement; and
   iii. provide the determined control response to the saltwater chlorinator.
O. A method comprising:
   i. receiving or obtaining a water hardness measurement; and
   ii. generating an output response based on the water hardness measurement.
P. The method of any preceding or subsequent statement or combination of statements, wherein generating the output response comprises controlling an operating parameter of a piece of pool equipment.
Q. The method of any preceding or subsequent statement or combination of statements, wherein generating the output response comprises controlling a maintenance parameter of a piece of pool equipment.
R. The method of any preceding or subsequent statement or combination of statements, wherein generating the output response comprises generating an alert or notification to a user.
S. A non-transitory computer readable storage medium comprising a plurality of instructions executable by one or more processors, the plurality of instructions comprising instructions which, when executed by the one or more processors, cause the one or more processors to perform actions including:
   i. receiving or obtaining a water hardness measurement; and
   ii. generating an output response based on the water hardness measurement.
T. The non-transitory computer readable medium of any preceding or subsequent statement or combination of statements, wherein the instructions cause the one or more processors to perform actions including the method of any preceding or subsequent statement or combination of statements.

These examples are not intended to be mutually exclusive, exhaustive, or restrictive in any way, and the invention is not limited to these example embodiments but rather encompasses all possible modifications and variations within the scope of any claims ultimately drafted and issued in connection with the invention (and their equivalents). For avoidance of doubt, any combination of features not physically impossible or expressly identified as non-combinable herein may be within the scope of the invention. Further, although applicant has described devices and techniques for use principally with swimming pools or spas, persons skilled in the relevant field will recognize that the present invention conceivably could be employed in connection with other water containing vessels and in other manners, particularly but not limited to underwater installations. Finally, references to “pools” and “swimming pools” herein may also refer to spas or other water containing vessels used for recreation, training, or therapy.

The subject matter of embodiments is described herein with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described. Directional references such as “up,” “down,” “top,” “bottom,” “left,” “right,” “front,” and “back,” among others, are intended to refer to the orientation as illustrated and described in the figure (or figures) to which the components and directions are referencing. Throughout this disclosure, a reference numeral with a letter refers to a specific instance of an element and the reference numeral without an accompanying letter refers to the element generically or collectively. Thus, as an example (not shown in the drawings), device “102A” refers to an instance of a device class, which may be referred to collectively as devices “102” and any one of which may be referred to generically as a device “102”. In the figures and the description, like numerals are intended to represent like elements. As used herein, the meaning of “a,” “an,” and “the” includes singular and plural references unless the context clearly dictates otherwise.

The above-described aspects are merely possible examples of implementations, merely set forth for a clear understanding of the principles of the present disclosure. Many variations and modifications can be made to the above-described embodiment(s) without departing substantially from the spirit and principles of the present disclosure. All such modifications and variations are intended to be included herein within the scope of the present disclosure, and all possible claims to individual aspects or combinations of elements or steps are intended to be supported by the present disclosure. Moreover, although specific terms are employed herein, as well as in the claims that follow, they are used only in a generic and descriptive sense, and not for the purposes of limiting the described invention, nor the claims that follow.

That which is claimed:

1. A water quality monitoring system comprising a water sensing device inline with a pool or spa and configured to automatically measure one or more water parameters using reagent, wherein the one or more water parameters comprises at least water hardness.

2. The water quality monitoring system of claim 1, wherein the water hardness comprises calcium hardness.

3. The water quality monitoring system of claim 1, wherein the water hardness comprises total hardness.

4. The water quality monitoring system of claim 1, wherein the reagent comprises liquid reagent.

5. The water quality monitoring system of claim 1, wherein the water sensing device is inline with the pool or spa such that water from the pool or spa flows through the water sensing device.

6. A water quality monitoring system configured to:
receive and/or obtain a water hardness measurement; and
automatically generate an output response based on the water hardness measurement.

7. The water quality monitoring system of claim 6, wherein the output response comprises an alert or notification on a user device.

8. The water quality monitoring system of claim 6, wherein the output response comprises control of a maintenance parameter of a piece of pool equipment.

9. The water quality monitoring system of claim 6, wherein the output response comprises control of an operating parameter of a piece of pool equipment.

10. The water quality monitoring system of statement 9, wherein the pool equipment comprises a saltwater chlorinator cell.

11. The water quality monitoring system of claim 9, wherein the one or more water parameters comprises calcium hardness.

12. The water quality monitoring system of claim 9, wherein the one or more water parameters comprises total hardness.

13. A water quality monitoring system comprising:
a water sensing device configured to automatically measure at least water hardness; and
a saltwater chlorinator, wherein the saltwater chlorinator is automatically controlled based on a water hardness measurement from the water sensing device.

14. The water quality monitoring system of claim 13, wherein the saltwater chlorinator is automatically controlled by controlling a polarity reversal frequency of a saltwater chlorinator cell of the saltwater chlorinator.

15. The water quality monitoring system of claim 13, wherein the water sensing device and the saltwater chlorinator are configured to directly communicate with each other.

16. The water quality monitoring system of claim 13, further comprising a control system communicatively connected to the water sensing device and the saltwater chlorinator, wherein the control system is configured to:
receive the water hardness measurement from the water sensing device;
determine a control response for the saltwater chlorinator based on the received water hardness measurement; and
provide the determined control response to the saltwater chlorinator.

17. The water quality monitoring system of claim 16, wherein the control response comprises an alert or notification on a user device.

18. The water quality monitoring system of claim 16, wherein the control response comprises control of a maintenance parameter of a piece of pool equipment.

19. The water quality monitoring system of claim 16, wherein the output response comprises control of an operating parameter of a piece of pool equipment.

20. The water quality monitoring system of claim 13, wherein the water sensing device is configured to automatically measure both the calcium hardness and the total hardness.
